(19) United States
(12) Patent Application Publication
(10) Pub. No.: US 2010/0093770 A1
(43) Pub. Date: Apr. 15, 2010

(54) THERAPEUTIC AGENT FOR GLAUCOMA CONTAINING ADENOSINE DERIVATIVE AS ACTIVE INGREDIENT

(76) Inventors: Atsushi Shimazaki, Ikoma-shi (JP); Noriko Kawabata, Ikoma-shi (JP); Tomoko Kirihara, Ikoma-shi (JP); Jayson M. Rieger, Charlottesville, VA (US); Robert D. Thompson, Charlottesville, VA (US)

Correspondence Address: FRISHAUF, HOLTZ, GOODMAN & CHICK, PC, 220 Fifth Avenue, 16TH Floor, NEW YORK, NY, 10001-7708, US

(21) Appl. No.: 12/450,832

(22) PCT Filed: Apr. 14, 2008
(86) PCT No.: PCT/US08/04770

§ 371 Date: Dec. 4, 2009

(30) Foreign Application Priority Data
Apr. 16, 2007 (JP) 2007-106915

(51) Int. Cl. A61K 31/52; A61P 27/06

(52) U.S. Cl. 514/263.23

(57) ABSTRACT

A method for treating glaucoma or ocular hypertension comprising administering to a patient a pharmacologically effective amount of a compound represented by the following (1) or a salt thereof:

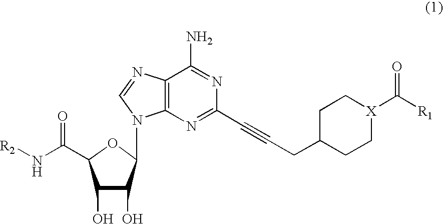

wherein X represents CH or N; R₁ represents a hydrogen atom, a hydroxy group, a halogen atom, an alkyl group, an alkoxy group, a cycloalkyl group, a cycloalkoxy group, a (cycloalkyl)methoxy group, or

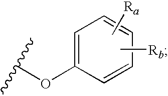

R₂ represents a hydrogen atom, an alkyl group, a cycloalkyl group, an alkylcarbonyl group or an alkyloxycarbonyl group; and Rₐ and R_{b} are the same or different and represent a hydrogen atom, a hydroxy group, a halogen atom, an alkyl group, an alkoxy group, a cycloalkyl group or a cycloalkoxy group.

### TECHNICAL FIELD

The present invention relates to a preventive or therapeutic agent for glaucoma or ocular hypertension containing a compound represented by the following general formula (1) or a salt thereof as an active ingredient.

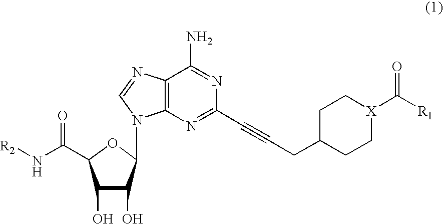

In the formula,

X represents CH or N;

R₁ represents a hydrogen atom, a hydroxy group, a halogen atom, an alkyl group, an alkoxy group, a cycloalkyl group, a cycloalkoxy group, a (cycloalkyl)alkoxy group, or

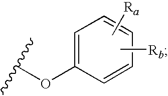

R₂ represents a hydrogen atom, an alkyl group, a cycloalkyl group, an alkylcarbonyl group or an alkyloxycarbonyl group; and

Rₐ and R_{b} are the same or different and represent a hydrogen atom, a hydroxy group, a halogen atom, an alkyl group, an alkoxy group, a cycloalkyl group or a cycloalkoxy group.

### BACKGROUND ART

Glaucoma is an intractable eye disease which exhibits increased intraocular pressure due to a variety of factors and involves a risk of leading to blindness. It is known that the incidence rate of glaucoma increases with age, and the progression of optic nerve injury also accelerates with age.

In US Patent Publication No. 2006-0100169, WO 2006/015357, WO 2006/101920 and Neuroscience, 141, 2029-2039 (2006), 4-{3-[6-amino-9-((2R,3R,4S,5S)-5-cyclopropylcarbamoyl-3,4-dihydroxytetrahydrofuran-2-yl)-9H-purin-2-yl]-2-propynyl}-piperidine-1-carboxylic acid methyl ester, which is a compound represented by the general formula (1), is disclosed and the compound is suggested to be useful as an anti-inflammatory agent, a coronary artery vasodilator, a neuroprotective agent or the like.

In WO 03/029264 and Neuroscience, 141, 2029-2039 (2006), 4-{3-[6-amino-9-((2R,3R,4S,5S)-5-ethylcarbamoyl-3,4-dihydroxytetrahydrofuran-2-yl)-9H-purin-2-yl]-2-propynyl}-piperidine-1-carboxylic acid methyl ester and 4-{3-[6-amino-9-((2R,3R,4S,5S)-5-ethylcarbamoyl-3,4-dihydroxytetrahydrofuran-2-yl)-9H-purin-2-yl]-2-propynyl}-piperidine-1-carboxylic acid isobutyl ester, both of which are a compound represented by the general formula (1), are disclosed, and in JP-T-2002-536300, 4-{3-[6-amino-9-((2R,3R,4S,5S)-5-ethylcarbamoyl-3,4-dihydroxytetrahydrofuran-2-yl)-9H-purin-2-yl]-2-propynyl}-cyclohexane-1-carboxylic acid methyl ester, which is a compound represented by the general formula (1), is disclosed. Further, in these documents, these compounds are suggested to be useful as an anti-inflammatory agent.

However, in reports related to the compound represented by the general formula (1), there is no report in which a pharmacological action of the compound on glaucoma or ocular hypertension, and further, there is no suggestion at all as to what type of adenosine derivative with what structure has an intraocular pressure lowering action.

### DISCLOSURE OF THE INVENTION

### Problems to be Solved

Accordingly, it is a very interesting subject to search a new medicinal use of a compound represented by the general formula (1).

### Means for Solving the Problems

The present inventors have made intensive studies in order to search a new medicinal use of a compound represented by the general formula (1) or a salt thereof (hereinafter these are also collectively referred to as the “present compound”), and as a result, they found that the present compound exhibits an excellent intraocular pressure lowering effect in a test for intraocular pressure reduction, and thus the present invention has been accomplished. Further, in the test, it was found that the present compound has a tendency to show lowering of trough intraocular pressure value (an intraocular pressure value before the subsequent administration is carried out in repeated administration) by repeated administration, and in particular, Compound A shows a high lowering action. That is, the present compound has a tendency to enhance the intraocular pressure lowering action by repeated administration and also shows excellent prolongation of efficacy.

That is, the present invention is directed to a preventive or therapeutic agent for glaucoma or ocular hypertension containing a compound represented by the general formula (1) or a salt thereof as an active ingredient.

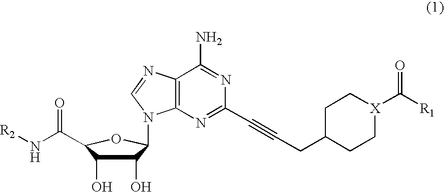

In the formula,

X represents CH or N;

R₁ represents a hydrogen atom, a hydroxy group, a halogen atom, an alkyl group, an alkoxy group, a cycloalkyl group, a cycloalkoxy group, a (cycloalkyl)alkoxy group, or

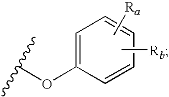

R₂ represents a hydrogen atom, an alkyl group, a cycloalkyl group, an alkylcarbonyl group or an alkyloxycarbonyl group; and

Rₐ and R_{b} are the same or different and represent a hydrogen atom, a hydroxy group, a halogen atom, an alkyl group, an alkoxy group, a cycloalkyl group or a cycloalkoxy group.

Further, another embodiment of the present invention is a method for preventing or treating glaucoma or ocular hypertension comprising administering a pharmacologically effective amount of a compound represented by the following general formula (1) or a salt thereof as an active ingredient to a patient.

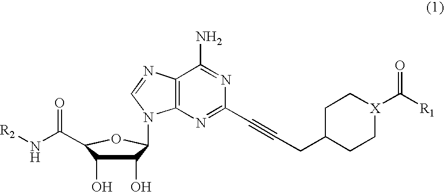

In the formula,

X represents CH or N;

R₁ represents a hydrogen atom, a hydroxy group, a halogen atom, an alkyl group, an alkoxy group, a cycloalkyl group, a cycloalkoxy group, a (cycloalkyl)alkoxy group, or

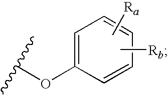

R₂ represents a hydrogen atom, an alkyl group, a cycloalkyl group, an alkylcarbonyl group or an alkyloxycarbonyl group; and

Rₐ and R_{b} are the same or different and represent a hydrogen atom, a hydroxy group, a halogen atom, an alkyl group, an alkoxy group, a cycloalkyl group or a cycloalkoxy group.

Further, another embodiment of the present invention is a compound represented by the following general formula (1) or a salt thereof for preventing or treating glaucoma or ocular hypertension.

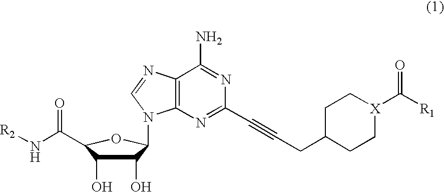

In the formula,

X represents CH or N;

R₁ represents a hydrogen atom, a hydroxy group, a halogen atom, an alkyl group, an alkoxy group, a cycloalkyl group, a cycloalkoxy group, a (cycloalkyl)alkoxy group, or

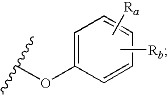

R₂ represents a hydrogen atom, an alkyl group, a cycloalkyl group, an alkylcarbonyl group or an alkyloxycarbonyl group; and

Rₐ and R_{b} are the same or different and represent a hydrogen atom, a hydroxy group, a halogen atom, an alkyl group, an alkoxy group, a cycloalkyl group or a cycloalkoxy group.

Further, another embodiment of the present invention is use of a compound represented by the following general formula (1) or a salt thereof for producing a preventive or therapeutic agent for glaucoma or ocular hypertension.

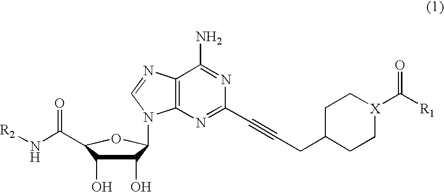

In the formula,

X represents CH or N;

R₁ represents a hydrogen atom, a hydroxy group, a halogen atom, an alkyl group, an alkoxy group, a cycloalkyl group, a cycloalkoxy group, a (cycloalkyl)alkoxy group, or

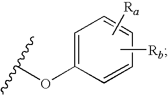

R₂ represents a hydrogen atom, an alkyl group, a cycloalkyl group, an alkylcarbonyl group or an alkyloxycarbonyl group; and

Rₐ and R_{b} are the same or different and represent a hydrogen atom, a hydroxy group, a halogen atom, an alkyl group, an alkoxy group, a cycloalkyl group or a cycloalkoxy group.

The respective groups as used in the claims and specification have the following meanings throughout the claims and specification.

The “halogen atom” refers to fluorine, chlorine, bromine or iodine.

The “alkyl” refers to linear or branched alkyl having 1 to 6 carbon atoms. Specific examples thereof include methyl, ethyl, n-propyl, n-butyl, n-pentyl, n-hexyl, isopropyl, isobutyl, sec-butyl, tert-butyl, isopentyl and the like.

The “cycloalkyl” refers to cycloalkyl having 3 to 8 carbon atoms. Specific examples thereof include cyclopropyl, cyclobutyl, cyclopentyl, cyclohexyl, cycloheptyl, cyclooctyl and the like.

The “alkoxy” refers to linear or branched alkoxy having 1 to 6 carbon atoms. Specific examples thereof include methoxy, ethoxy, n-propoxy, n-butoxy, n-pentoxy, n-hexyloxy, isopropoxy, isobutoxy, sec-butoxy, tert-butoxy, isopentyloxy and the like.

The “cycloalkoxy” refers to cycloalkoxy having 3 to carbon atoms. Specific examples thereof include cyclopropoxy, cyclobutoxy, cyclopentoxy, cyclohexyloxy, cycloheptyloxy, cyclooctyloxy and the like.

The “(cycloalkyl)alkoxy” refers to cycloalkyl having 3 to 8 carbon atoms and alkoxy as defined above. Specific examples thereof include (cyclopropyl)methoxy, (cyclobutyl)methoxy, (cyclopentyl)methoxy, (cyclohexyl)methoxy, (cycloheptyl)methoxy, (cyclooctyl)methoxy and the like.

The “alkylcarbonyl” refers to linear or branched alkylcarbonyl having 2 to 7 carbon atoms. Specific examples thereof include methylcarbonyl, ethylcarbonyl, n-propylcarbonyl, n-butylcarbonyl, n-pentylcarbonyl, n-hexylcarbonyl, isopropylcarbonyl, isobutylcarbonyl, sec-butylcarbonyl, tert-butylcarbonyl, isopentylcarbonyl and the like.

The “alkyloxycarbonyl” refers to linear or branched alkyloxycarbonyl having 2 to 7 carbon atoms. Specific examples thereof include methoxycarbonyl, ethoxycarbonyl, n-propoxycarbonyl, n-butoxycarbonyl, n-pentoxycarbonyl, n-hexyloxycarbonyl, isopropoxycarbonyl, isobutoxycarbonyl, sec-butoxycarbonyl, tert-butoxycarbonyl, isopentoxycarbonyl and the like.

The “salt” of the present compound is not particularly limited as long as it is a pharmaceutically acceptable salt, and examples thereof include salts with an inorganic acid such as hydrochloric acid, hydrobromic acid, hydroiodic acid, nitric acid, sulfuric acid or phosphoric acid; salts with an organic acid such as acetic acid, fumalic acid, maleic acid, succinic acid, citric acid, tartaric acid, adipic acid, gluconic acid, glucoheptonic acid, glucuronic acid, terephthalic acid, methanesulfonic acid, lactic acid, hippuric acid, 1,2-ethanedisulfonic acid, isethionic acid, lactobionic acid, oleic acid, pamoic acid, polygalacturonic acid, stearic acid, tannic acid, trifluoromethanesulfonic acid, benzenesulfonic acid, p-toluenesulfonic acid, lauryl sulfate, methyl sulfate, naphthalenesulfonic acid or sulfosalicylic acid; quaternary ammonium salts such as methyl bromide, methyl iodide; salts with a halogen ion such as a bromine ion, a chlorine ion or an iodine ion; salts with an alkali metal such as lithium, sodium or potassium; salts with an alkaline earth metal such as calcium or magnesium; salts with a metal such as iron or zinc; salts with ammonia; salts with an organic amine such as triethylenediamine, 2-aminoethanol, 2,2-iminobis(ethanol), 1-deoxy-1-(methylamino)-2-D-sorbitol, 2-amino-2-(hydroxymethyl)-1,3-propanediol, procaine or N,N-bis(phenylmethyl)-1,2-ethanediamine, and the like.

In the case where there are geometrical isomers or optical isomers in the present compound, these isomers are also included in the scope of the present invention.

Further, the present compound may be in the form of a hydrate or a solvate. Further, in the case where there is tautomerism or polymorphism in the present compound, these compounds are also included in the scope of the present invention.

(a) Preferred examples of the present compound include compounds in which the respective groups are as defined below in the compounds represented by the general formula (1) and salts thereof.

(a1) X represents CH or N; and/or

(a2) R₁ represents a hydroxy group, an alkoxy group, a cycloalkoxy group, a (cycloalkyl)alkoxy group, or

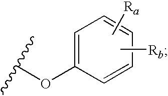

and/or

(a3) R₂ represents an alkyl group or a cycloalkyl group; and/or

(a4) Rₐ and R_{b} are the same or different and represent a hydrogen atom, a halogen atom, or an alkoxy group.

That is, in the compounds represented by the general formula (1), preferred examples include compounds that comprise one or each combination of two or more selected from the above (a1), (a2), (a3), and (a4), and salts thereof.

(b) More preferred examples of the present compound include compounds in which the respective groups are as defined below in the compounds represented by the general formula (1) and salts thereof.

(b1) X represents CH or N; and/or

(b2) R₁ represents a methoxy group, an ethoxy group, an isopropoxy group, an isobutoxy group, a cyclobutoxy group, a (cyclopropyl)alkoxy group, a 4-fluorophenyloxy group, a 2-methoxyphenyloxy group, a 4-methoxyphenyloxy group or a 3,4-difluorophenyloxy group; and/or

(b3) R₂ represents an ethyl group or a cyclopropyl group.

That is, in the compounds represented by the general formula (1), preferred examples include compounds that comprise one or each combination of two or more selected from the above (b1), (b2), and (b3), and salts thereof.

Most preferred examples of the present compound include:
4-{3-[6-amino-9-((2R,3R,4S,5S)-5-cyclopropylcarbamoyl-3,4-dihydroxytetrahydrofuran-2-yl)-9H-purin-2-yl]-2-propynyl}-piperidine-1-carboxylic acid methyl ester represented by the following formula (2);
4-{3-[6-amino-9-((2R,3R,4S,5S)-5-ethylcarbamoyl-3,4-dihydroxytetrahydrofuran-2-yl)-9H-purin-2-yl]-2-propynyl}-piperidine-1-carboxylic acid methyl ester represented by the following formula (3);
4-{3-[6-amino-9-((2R,3R,4S,5S)-5-ethylcarbamoyl-3,4-dihydroxytetrahydrofuran-2-yl)-9H-purin-2-yl]-2-propynyl}-piperidine-1-carboxylic acid isobutyl ester represented by the following formula (4);
4-{3-[(6-amino-9-((2R,3R,4S,5S)-5-ethylcarbamoyl-3,4-dihydroxytetrahydrofuran-2-yl)-9H-purin-2-yl]-2-propynyl}-cyclohexane-1-carboxylic acid methyl ester represented by the following formula (5);
4-{3-[6-amino-9-((2R,3R,4S,5S)-5-ethylcarbamoyl-3,4-dihydroxytetrahydrofuran-2-yl)-9H-purin-2-yl]-2-propynyl}-piperidine-1-carboxylic acid ethyl ester represented by the following formula (6);
4-{3-[6-amino-9-((2R,3R,4S,5S)-5-ethylcarbamoyl-3,4-dihydroxytetrahydrofuran-2-yl)-9H-purin-2-yl]-2-propynyl}-piperidine-1-carboxylic acid isopropyl ester represented by the following formula (7);
4-{3-[6-amino-9-((2R,3R,4S,5S)-5-cyclopropylcarbamoyl-3,4-dihydroxytetrahydrofuran-2-yl)-9H-purin-2-yl]-2-propynyl}-piperidine-1-carboxylic acid 4-fluorophenyl ester represented by the following formula (8);
4-{3-[6-amino-9-((2R,3R,4S,5S)-5-cyclopropylcarbamoyl-3,4-dihydroxytetrahydrofuran-2-yl)-9H-purin-2-yl]-2-propynyl}-piperidine-1-carboxylic acid 2-methoxyphenyl ester represented by the following formula (9);
4-{3-[6-amino-9-((2R,3R,4S,5S)-5-cyclopropylcarbamoyl-3,4-dihydroxytetrahydrofuran-2-yl)-9H-purin-2-yl]-2-propynyl}-piperidine-1-carboxylic acid 4-methoxyphenyl ester represented by the following formula (10);
4-{3-[6-amino-9-((2R,3R,4S,5S)-5-cyclopropylcarbamoyl-3,4-dihydroxytetrahydrofuran-2-yl)-9H-purin-2-yl]-2-propynyl}-piperidine-1-carboxylic acid 3,4-difluorophenyl ester represented by the following formula (11); and
4-{3-[6-amino-9-((2R,3R,4S,5S)-5-cyclopropylcarbamoyl-3,4-dihydroxytetrahydrofuran-2-yl)-9H-purin-2-yl]-2-propynyl}-piperidine-1-carboxylic acid cyclobutyl ester represented by the following formula (12).

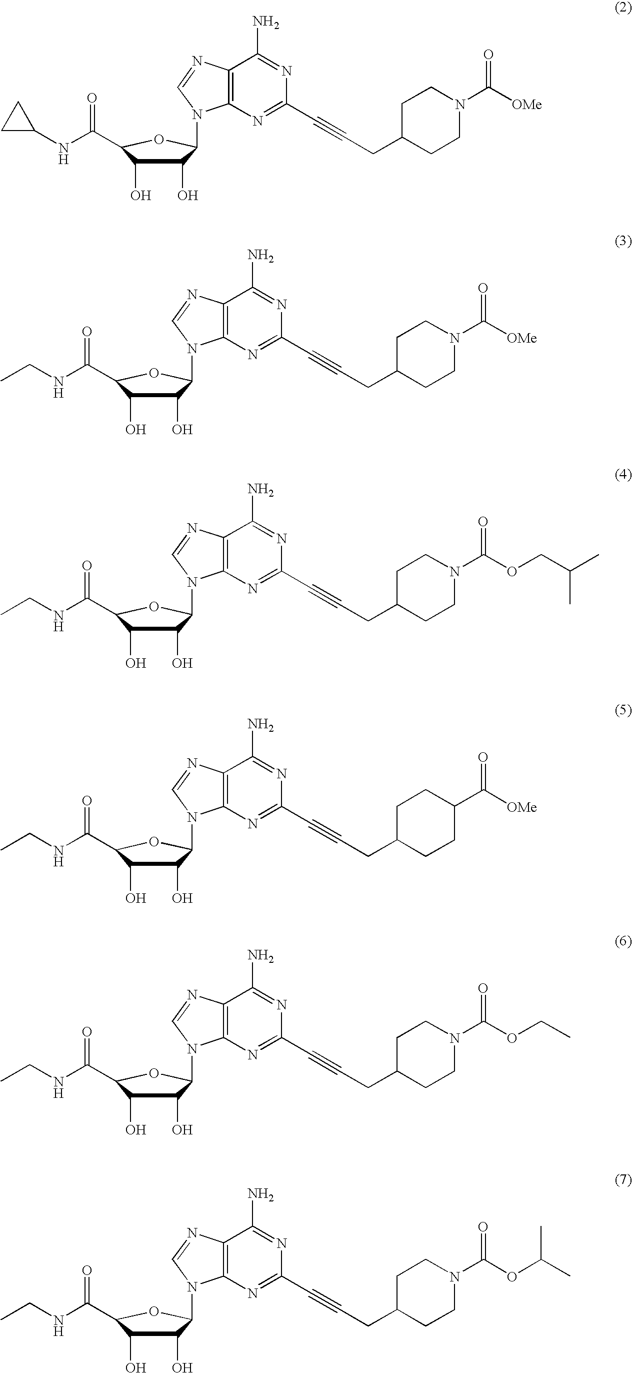

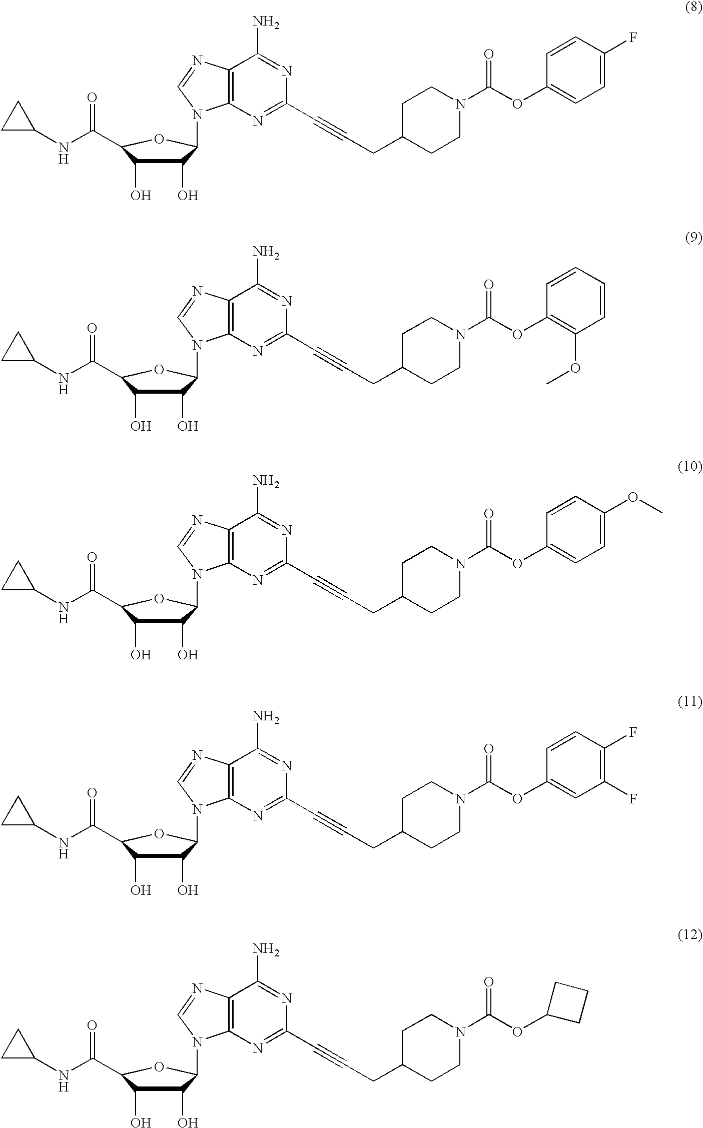

The present compound can be produced according to a common procedure in the field of organic synthetic chemistry, and also can be produced based on the method described in WO2003/029264, JP-T-2005-508933, WO 2006/015357, WO 2007/136817 or JP-T-2002-536300.

The compound of formula (12) can be prepared as follows.

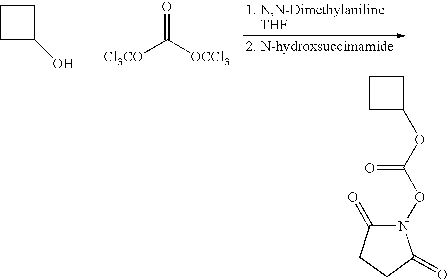

To triphosgene (0.34 eq) stirring in THF at 0° C. under inert atmosphere, the alcohol (1.0 eq) and dimethylaniline (1.1 eq) are added slowly as a solution in dry THF. After ten minutes, the reaction is warmed to room temperature and stirred for an additional 3 h. Dry DCM is then added and the mixture is poured slowly into a solution of N-hydroxysuccinamide (1.3 eq) in dry DCM at 0° C. The reaction is slowly warmed to room temperature and stirred overnight. Water is added to the mixture and after stirring for an additional 3 h, the solution is diluted with EtOAc. The organic layer is washed 3 times with water, once with brine, then dried (MgSO₄) and concentrated. The resulting oil (which may be a mixture of the carbonate and symmetrical anhydride) was taken directly onto the next step.

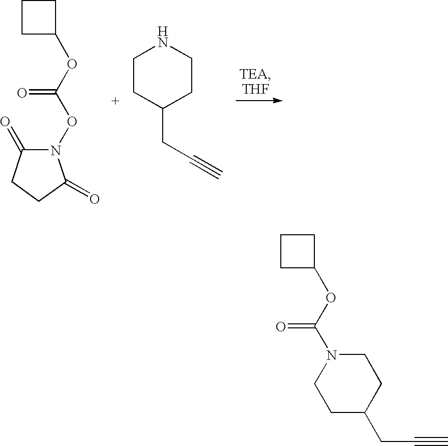

The piperidine derivative (0.75 eq) is dissolved in dry THF and TEA (excess) is added slowly at room temperature under inert atmosphere. The carbonate compound (1.0 eq) is diluted with THF and added dropwise to the piperidine solution. The mixture is stirred for 24 h then concentrated for application to silica gel chromatography (gradient starting at 100% hexanes up to 80% DCM in hexanes). The resulting oil (˜60% yield) is stored at 4° C. until further use.

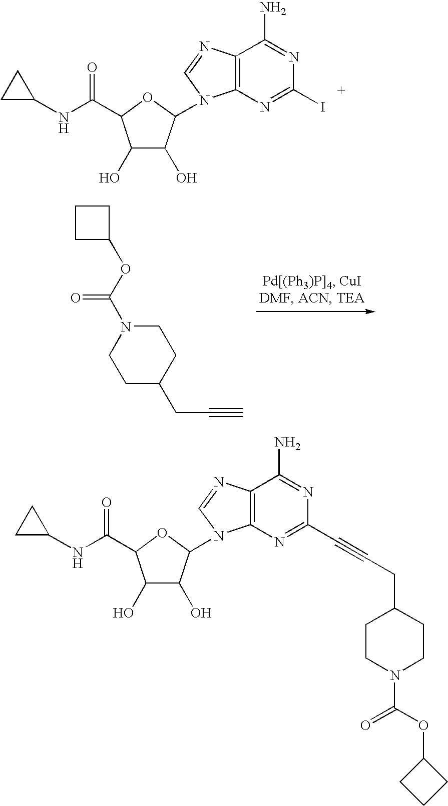

Iodo derivative (1.0 eq) is dissolved in a solution of DMF:ACN:TEA 5:5:1 (all solvent vigorously degassed) and stirred at room temperature under inert atmosphere. Palladium catalyst (˜5 mol %) and copper [I] iodide (1.05 eq) are added followed by the alkyne derivative (4.0 eq). The resulting dark solution is stirred overnight then concentrated for application to silica gel chromatography (gradient starting at 100% DCM up to 10% MeOH in DCM). The resulting oil was further purified by preparative HPLC to obtain an off white solid (˜30% yield).

¹H NMR (DDMSO) δ 8.56 (s, 1H), 8.30 (s, 1H), 7.52 (s, 2H), 5.97 (d, 1H, J=6.6), 5.67 (dd, 2H, J=21.3, 4.8), 4.84 (p, 1H, J=5.9), 4.64 (q, 1H, J=4.8), 4.30 (d, 1H, J=2.1), 4.21 (m, 1H), 4.00 (d, 2H, J=12.9), 3.12 (m, 1H), 2.719 (m, 4H), 2.430 (d, 2H, J=6.3), 2.272 (m, 2H), 2.00 (m, 2H), 1.77 (m, 2H), 1.56 (m, 2H), 1.207 (m, 2H), 0.68 (m, 1H), 0.50 (m, 1H). LRMS ESI (M+H⁺) 540.35. HPLC: MeOH 20-95% gradient in water over 4 minutes at 40° C., 6 minutes total. Retention Time=3.04 min (6 min method).

The preventive or therapeutic agent for glaucoma or ocular hypertension of the present invention can be administered either orally or parenterally.

Examples of the dosage form include eye drops, ophthalmic ointments, injections, tablets, capsules, granules, powders and the like. In particular, eye drops are preferred. These can be prepared using any, of generally used techniques. For example, in the case of eye drops, a desired eye drop can be prepared by adding the present compound to purified water or a buffer or the like, stirring the mixture, and then adjusting the pH of the solution with a pH adjusting agent. Further, an additive which is generally used in eye drops can be used as needed. For example, formulation thereof can be carried out using a tonicity agent such as sodium chloride or concentrated glycerin, a buffer such as sodium phosphate, sodium acetate, boric acid, borax or citric acid, a surfactant such as polyoxyethylene sorbitan monooleate, polyoxyl stearate or polyoxyethylene hydrogenated castor oil, a stabilizer such as sodium citrate or sodium edetate, a preservative such as benzalkonium chloride or paraben, and the like. The pH of the eye drops is permitted as long as it falls within the range that is acceptable as an ophthalmic preparation, but is preferably in the range of from 3 to 8.

The ophthalmic ointments can be prepared with a generally used base such as white petrolatum or liquid paraffin. Also, oral preparations such as tablets, capsules, granules and powders can be prepared by adding an extender such as lactose, crystalline cellulose, starch or vegetable oil, a lubricant such as magnesium stearate or talc, a binder such as hydroxypropyl cellulose or polyvinyl pyrrolidone, a disintegrant such as carboxymethyl cellulose calcium or low-substituted hydroxypropylmethyl cellulose, a coating agent such as hydroxypropylmethyl cellulose, macrogol or a silicone resin, a film forming agent such as gelatin film, and the like, as needed.

The dose of the present compound can properly be changed depending on the dosage form, severity of symptoms, age, body weight of a patient to be administered, doctor's judgment, and the like. In the case of an eye drop, an eye drop containing an active ingredient at a concentration of generally from 0.000001 to 10% (w/v), preferably from 0.00001 to 3% (w/v), more preferably 0.0001 to 1% (w/v), further more preferably 0.001 to 0.1% (w/v) may be instilled to an adult once to several times a day. In the case of oral administration, the present compound may be administered to an adult once or divided into several times at a dose of generally from 0.01 to 5000 mg per day, preferably from 0.1 to 2500 mg per day, more preferably from 1 to 1000 mg per day.

### ADVANTAGE OF THE INVENTION

As will be described in detail in the section of pharmacological test below, when a test for intraocular pressure reduction was carried out using cynomolgus monkeys or Japanese white rabbits, it was shown that
4-{3-[6-amino-9-((2R,3R,4S,5S)-5-cyclopropylcarbamoyl-3,4-dihydroxytetrahydrofuran-2-yl)-9H-purin-2-yl]-2-propynyl}-piperidine-1-carboxylic acid methyl ester (hereinafter also referred to as “Compound A”),
4-{3-[6-amino-9-((2R,3R,4S,5S)-5-ethylcarbamoyl-3,4-dihydroxytetrahydrofuran-2-yl)-9H-purin-2-yl]-2-propynyl}-cyclohexane-1-carboxylic acid methyl ester (hereinafter also referred to as “Compound B”),
4-{3-[6-amino-9-((2R,3R,4S,5S)-5-ethylcarbamoyl-3,4-dihydroxytetrahydrofuran-2-yl)-9H-purin-2-yl]-2-propynyl}-piperidine-1-carboxylic acid methyl ester (hereinafter also referred to as “Compound C”),
4-{3-[6-amino-9-((2R,3R,4S,5S)-5-ethylcarbamoyl-3,4-dihydroxytetrahydrofuran-2-yl)-9H-purin-2-yl]-2-propynyl}-piperidine-1-carboxylic acid ethyl ester (hereinafter also referred to as “Compound D”),
4-{3-[6-amino-9-((2R,3R,4S,5S)-5-ethylcarbamoyl-3,4-dihydroxytetrahydrofuran-2-yl)-9H-purin-2-yl]-2-propynyl}-piperidine-1-carboxylic acid isopropyl ester (hereinafter also referred to as “Compound E”),
4-{3-[6-amino-9-((2R,3R,4S,5S)-5-cyclopropylcarbamoyl-3,4-dihydroxytetrahydrofuran-2-yl)-9H-purin-2-yl]-2-propynyl}-piperidine-1-carboxylic acid 4-fluorophenyl ester (hereinafter also referred to as “Compound F”),
4-{3-[(6-amino-9-((2R,3R,4S,5S)-5-cyclopropylcarbamoyl-3,4-dihydroxytetrahydrofuran-2-yl)-9H-purin-2-yl]-2-propynyl}-piperidine-1-carboxylic acid 2-methoxyphenyl ester (hereinafter also referred to as “Compound G”),
4-{3-[6-amino-9-((2R,3R,4S,5S)-5-cyclopropylcarbamoyl-3,4-dihydroxytetrahydrofuran-2-yl)-9H-purin-2-yl]-2-propynyl}-piperidine-1-carboxylic acid 4-methoxyphenyl ester (hereinafter also referred to as “Compound H”),
4-{3-[6-amino-9-((2R,3R,4S,5S)-5-cyclopropylcarbamoyl-3,4-dihydroxytetrahydrofuran-2-yl)-9H-purin-2-yl]-2-propynyl}-piperidine-1-carboxylic acid 3,4-difluorophenyl ester (hereinafter also referred to as “Compound I”), and
4-{3-[6-amino-9-((2R,3R,4S,5S)-5-cyclopropylcarbamoyl-3,4-dihydroxytetrahydrofuran-2-yl)-9H-purin-2-yl]-2-propynyl}-piperidine-1-carboxylic acid cyclobutyl ester (hereinafter also referred to as “Compound J”) exhibit an excellent intraocular pressure lowering effect. That is, the present compounds are useful as a preventive or therapeutic agent for glaucoma or ocular hypertension.

### BEST MODE FOR CARRYING OUT THE INVENTION

Hereinafter, the results of pharmacological test and preparation examples will be described, however, these examples are described for the purpose of understanding the present invention better and are not meant to limit the scope of the present invention.

Pharmacological Test

### (1) Test for Intraocular Pressure Reduction Using Cynomolgus Monkeys

In order to examine the usefulness of the present compound as a preventive or therapeutic agent for glaucoma or ocular hypertension, an intraocular pressure lowering effect when the present compound was administered to cynomolgus monkeys (sex: male) was evaluated and studied. As the test compound, Compound A, Compound B, Compound C, Compound D, Compound E, Compound F, Compound G, Compound H and Compound I were used.

(Evaluation Test Method for Intraocular Pressure Reduction)

1) Just before a test liquid was administered, one drop of 0.4% oxybuprocaine hydrochloride eye drop was instilled into both eyes of each experimental animal to achieve local anesthesia, and the intraocular pressure was measured using an applanation tonometer. This intraocular pressure was determined as an initial intraocular pressure.

2) A 0.1% (w/v) test liquid (a solution or suspension) was prepared, and the test liquid was instilled into one eye of each experimental animal twice a day for 7 days. The other eye was left untreated, or a vehicle was instilled into the eye according to the same schedule. Incidentally, the test liquid was prepared according to the preparation method for an eye drop described above. Specifically, to 10 mM phosphate buffer or 1.7% borate buffer, polysorbate 80 and any of the test compounds were added and dissolved or dispersed therein. Then, the pH of the resulting solution or dispersion was adjusted to 5 with sodium hydroxide and/or dilute hydrochloric acid, whereby a test liquid containing each test compound was prepared (only in the case of Compound B, the pH of the test liquid was adjusted to 7).

3) After the test liquid was administered, the intraocular pressure of both eyes of each experimental animal was measured at predetermined times (at 2, 4, 6, and 8 hours after administration) (only in the case of Compound B, the intraocular pressure was measured at 1, 2, 4 and 6 hours after administration). Incidentally, before measurement, one drop of 0.4% oxybuprocaine hydrochloride eye drop was instilled into both eyes of each experimental animal to achieve local anesthesia.

(Calculation Formula for Intraocular Pressure Reduction Degree)

The intraocular pressure reduction degree of each test compound administration group at each measurement time was calculated from the following calculation formula. Among the obtained intraocular pressure reduction degrees at respective measurement times, the maximum value was determined as a maximum intraocular pressure reduction degree.

Intraocular pressure reduction degree (mmHg)=|IOP (D−t)−IOP (D−0)| Equation 1

IOP (D−t): Intraocular pressure of the eye into which the test compound was administered at t hours after administration of test compound
IOP (D−0): Initial intraocular pressure of the eye into which the test compound was administered

(Results and Discussion)

The test results (maximum intraocular pressure reduction degree (mmHg)) in the case of using Compound A, Compound B, Compound C, Compound D, Compound E, Compound F, Compound G, Compound H and Compound I are shown in Table 1. As is apparent from Table 1, every compound exhibited an excellent intraocular pressure lowering action. That is, it was found that the present compounds as typified by Compound A, Compound B and the like are particularly useful as a preventive or therapeutic agent for glaucoma or ocular hypertension. It was found that in particular, Compound A exhibited a significantly higher intraocular pressure lowering action among the present compounds. Further, the present compounds have a tendency to show lowering of trough intraocular pressure by repeated instillation (BID), and particularly Compound A exhibited a high lowering action. Specifically, in the case of Compound A, the trough intraocular pressure value on day 7 was lower by as much as 1.0 mmHg than that in the vehicle administration group, and Compound A exhibited a significant lowering action.

TABLE 1

| | Maximum intraocular pressure |
|---|---|
| Test Compound | reduction degree (mmHg) |
| Compound A | 4.9 |
| Compound B | 2.2 |
| Compound C | 2.7 |
| Compound D | 2.9 |
| Compound E | 2.9 |
| Compound F | 2.7 |
| Compound G | 2.7 |
| Compound H | 2.3 |
| Compound I | 1.8 |

*: Incidentally, the maximum intraocular pressure reduction degree is represented by the average value for each group consisting of 5 to 6 cases.

### (2) Test for Intraocular Pressure Reduction Using Japanese White Rabbits

In order to examine the usefulness of the present compound as a preventive or therapeutic agent for glaucoma or ocular hypertension, an intraocular pressure lowering effect when the present compound was administered to Japanese white rabbits (sex: male) was evaluated and studied. As the test compound, Compound A, Compound B, Compound C, Compound E, Compound I and Compound J were used.

(Preparation of Test Liquid)

According to the preparation method for an eye drop described above, each test liquid containing Compound A (0.003% (w/v)), Compound B (0.2% (w/v)), Compound C (0.01% (w/v)), Compound E (0.01% (w/v)), Compound I (0.01% (w/v)) or Compound J (0.01% (w/v)) was prepared. Specifically, to 10 mM phosphate buffer or 1.7% borate buffer, polysorbate 80 and any of the test compounds were added and dissolved or dispersed therein. Then, the pH of the resulting solution or dispersion was adjusted to 5 with sodium hydroxide and/or dilute hydrochloric acid, whereby a test liquid containing each test compound was prepared (only in the case of Compound B, the pH of the test liquid was adjusted to 7).

(Administration Method and Measurement Method)

1) Just before any of the test liquids was administered, one drop of 0.4% oxybuprocaine hydrochloride eye drop was instilled into both eyes of each experimental animal to achieve local anesthesia, and the intraocular pressure was measured using an applanation tonometer. This intraocular pressure was determined as an initial intraocular pressure.

2) Any of the prepared test liquids was administered into one eye of each experimental animal in a single dose. The other eye was left untreated, or a vehicle was instilled into the eye according to the same schedule.

3) After the test liquid was administered, the intraocular pressure of both eyes of each experimental animal was measured at predetermined times (at 1, 2, 4 and hours after administration). Incidentally, before measurement, one drop of 0.4% oxybuprocaine hydrochloride eye drop was instilled into both eyes of each experimental animal to achieve local anesthesia.

(Calculation of Intraocular Pressure Reduction Degree)

The intraocular pressure reduction degree of each test compound administration group at each measurement time was calculated from the following calculation formula.

Intraocular pressure reduction degree (mmHg)=|IOP (Ad−t)−IOP (Ad−0)| Equation 2

IOP (Ad−t): Intraocular pressure of the eye into which the test compound was administered at t hours after administration of test compound
IOP (Ad−0): Initial intraocular pressure of the eye into which the test compound was administered

(Results and Discussion)

The test results (intraocular pressure reduction degree (mmHg) at 2 or 4 hours after instillation at which the intraocular pressure decreased most) in the case of using Compound A, Compound B, Compound C, Compound E, Compound I and Compound J are shown in Table 2. As is apparent from Table 2, every compound exhibited an excellent intraocular pressure lowering action. That is, it was found that the present compounds as typified by Compound A, Compound B and the like are particularly useful as a preventive or therapeutic agent for glaucoma or ocular hypertension. It was found that in particular, Compound A and Compound J exhibited a significantly higher intraocular pressure lowering action among the present compounds.

TABLE 2

| | Intraocular pressure |
|---|---|
| Test Compound | reduction degree (mmHg) |
| Compound A | 3.8 |
| Compound B | 2.1 |
| Compound C | 2.8 |
| Compound E | 2.6 |
| Compound I | 1.7 |
| Compound J | 4.1 |

*: Incidentally, the intraocular pressure reduction degree is represented by the average value for each group consisting of 5 to 6 cases.

### PREPARATION EXAMPLES

Hereinafter, representative preparation examples using the present compound will be shown.

### Preparation Examples

A medicinal agent of the present invention will be more specifically described with reference to preparation examples, however, the invention is not limited only to these preparation examples.

### Formulation Example 1

### Eye Drop

In 100 ml,

| | |
|---|---|
| Compound A | 0.1 g |
| Concentrated glycerin | 2.6 g |
| Sodium dihydrogen phosphate | q.s. |
| Polysorbate 80 | q.s. |
| Sodium hydroxide | q.s. |
| Dilute hydrochloric acid | q.s. |
| Sterile purified water | q.s. |

To sterile purified water, Compound A and the other components described above are added, and these components are well mixed, whereby an eye drop is prepared. By changing the amount of Compound A to be added, an eye drop at a concentration of 0.01% (w/v), 0.03% (w/v), 0.05% (w/v), or 0.3% (w/v) can be prepared.

### Formulation Example 2

### Eye Drop

In 100 ml

| | |
|---|---|
| Compound B | 0.1 g |
| Boric acid | 2.0 g |
| Polysorbate 80 | q.s. |
| Sodium hydroxide | q.s. |
| Dilute hydrochloric acid | q.s. |
| Sterile purified water | q.s. |

To sterile purified water, Compound B and the other components described above are added, and these components are well mixed, whereby an eye drop is prepared. By changing the amount of Compound B to be added, an eye drop at a concentration of 0.01% (w/v), 0.03% (w/v), 0.05% (w/v), or 0.3% (w/v) can be prepared.

1. A composition for treating glaucoma or ocular hypertension comprising a pharmacologically effective amount of a compound represented by the following formula (1) or a salt thereof as an active ingredient: 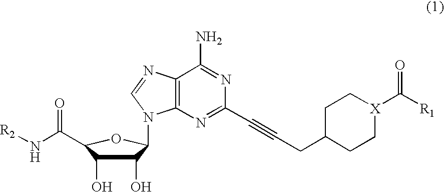
wherein
X represents CH or N;
R₁ represents a hydrogen atom, a hydroxy group, a halogen atom, an alkyl group, an alkoxy group, a cycloalkyl group, a cycloalkoxy group, a (cycloalkyl)alkoxy group, or 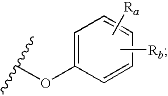
R₂ represents a hydrogen atom, an alkyl group, a cycloalkyl group, an alkylcarbonyl group or an alkyloxycarbonyl group; and
Rₐ and R_{b} are the same or different and represent a hydrogen atom, a hydroxy group, a halogen atom, an alkyl group, an alkoxy group, a cycloalkyl group or a cycloalkoxy group, and a pharmacologically acceptable ophthalmic carrier.

2. The composition according to claim 1, wherein in the formula (1),
X represents CH or N;
R₁ represents a hydroxy group, an alkoxy group, a cycloalkoxy group, a (cycloalkyl)alkoxy group, or 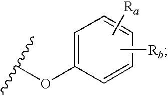
R₂ represents an alkyl group or a cycloalkyl group; and
Rₐ and R_{b} are the same or different and represent a hydrogen atom, a halogen atom or an alkoxy group.

3. The composition according to claim 1, wherein in the formula (1),
X represents CH or N;
R₁ represents a methoxy group, an ethoxy group, an isopropoxy group, an isobutoxy group, a cyclobutoxy group, a (cyclopropyl)methoxy group, a 4-fluorophenyloxy group, a 2-methoxyphenyloxy group, a 4-methoxyphenyloxy group or a 3,4-difluorophenyloxy group; and
R₂ represents an ethyl group or a cyclopropyl group.

4. The composition according to claim 1, wherein the compound represented by the formula (1) is selected from the group consisting of
4-{(3-[6-amino-9-((2R,3R,4S,5S)-5-cyclopropylcarbamoyl-3,4-dihydroxytetrahydrofuran-2-yl)-9H-purin-2-yl]-2-propynyl}-piperidine-1-carboxylic acid methyl ester;
4-{(3-[6-amino-9-((2R,3R,4S,5S)-5-ethylcarbamoyl-3,4-dihydroxytetrahydrofuran-2-yl)-9H-purin-2-yl]-2-propynyl}-piperidine-1-carboxylic acid methyl ester;
4-{3-[6-amino-9-((2R,3R,4S,5S)-5-ethylcarbamoyl-3,4-dihydroxytetrahydrofuran-2-yl)-9H-purin-2-yl]-2-propynyl}-piperidine-1-carboxylic acid isobutyl ester;
4-{3-[6-amino-9-((2R,3R,4S,5S)-5-ethylcarbamoyl-3,4-dihydroxytetrahydrofuran-2-yl)-9H-purin-2-yl]-2-propynyl}-cyclohexane-1-carboxylic acid methyl ester;
4-{3-[6-amino-9-((2R,3R,4S,5S)-5-ethylcarbamoyl-3,4-dihydroxytetrahydrofuran-2-yl)-9H-purin-2-yl]-2-propynyl}-piperidine-1-carboxylic acid ethyl ester;
4-{3-[6-amino-9-((2R,3R,4S,5S)-5-ethylcarbamoyl-3,4-dihydroxytetrahydrofuran-2-yl)-9H-purin-2-yl]-2-propynyl}-piperidine-1-carboxylic acid isopropyl ester;
4-{3-[6-amino-9-((2R,3R,4S,5S)-5-cyclopropylcarbamoyl-3,4-dihydroxytetrahydrofuran-2-yl)-9H-purin-2-yl]-2-propynyl}-piperidine-1-carboxylic acid 4-fluorophenyl ester;
4-{3-[6-amino-9-((2R,3R,4S,5S)-5-cyclopropylcarbamoyl-3,4-dihydroxytetrahydrofuran-2-yl)-9H-purin-2-yl]-2-propynyl}-piperidine-1-carboxylic acid 2-methoxyphenyl ester;
4-{3-[(6-amino-9-((2R,3R,4S,5S)-5-cyclopropylcarbamoyl-3,4-dihydroxytetrahydrofuran-2-yl)-9H-purin-2-yl]-2-propynyl}-piperidine-1-carboxylic acid 4-methoxyphenyl ester;
4-{3-[6-amino-9-((2R,3R,4S,5S)-5-cyclopropylcarbamoyl-3,4-dihydroxytetrahydrofuran-2-yl)-9H-purin-2-yl]-2-propynyl}-piperidine-1-carboxylic acid 3,4-difluorophenyl ester; and
4-{3-[6-amino-9-((2R,3R,4S,5S)-5-cyclopropylcarbamoyl-3,4-dihydroxytetrahydrofuran-2-yl)-9H-purin-2-yl]-2-propynyl}-piperidine-1-carboxylic acid cyclobutyl ester.

5. The composition according to claim 1, wherein the composition is in a dosage form which is an eye drop or an ophthalmic ointment.

6. A method for treating glaucoma or ocular hypertension, comprising administering to a patient a pharmacologically effective amount of a compound represented by the following formula (1) or a salt thereof as an active ingredient to a patient: 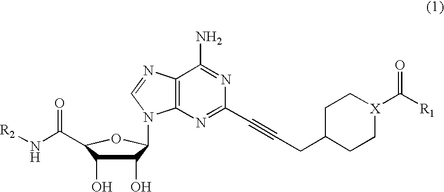
wherein
X represents CH or N;
R₁ represents a hydrogen atom, a hydroxy group, a halogen atom, an alkyl group, an alkoxy group, a cycloalkyl group, a cycloalkoxy group, a (cycloalkyl)alkoxy group, or 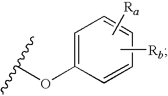
R₂ represents a hydrogen atom, an alkyl group, a cycloalkyl group, an alkylcarbonyl group or an alkyloxycarbonyl group; and
Rₐ and R_{b} are the same or different and represent a hydrogen atom, a hydroxy group, a halogen atom, an alkyl group, an alkoxy group, a cycloalkyl group or a cycloalkoxy group.

7. The method according to claim 6, wherein in the formula (1),
X represents CH or N;
R₁ represents a hydroxy group, an alkoxy group, a cycloalkoxy group, a (cycloalkyl)alkoxy group, or 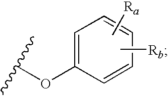
R₂ represents an alkyl group or a cycloalkyl group; and
Rₐ and R_{b} are the same or different and represent a hydrogen atom, a halogen atom, or an alkoxy group.

8. The method according to claim 6, wherein in the formula (1),
X represents CH or N;
R₁ represents a methoxy group, an ethoxy group, an isopropoxy group, an isobutoxy group, a cyclobutoxy group, a (cyclopropyl)methoxy group, a 4-fluorophenyloxy group, a 2-methoxyphenyloxy group, a 4-methoxyphenyloxy group or a 3,4-difluorophenyloxy group; and
R₂ represents an ethyl group or a cyclopropyl group.

9. The method according to claim 6, wherein the compound represented by the formula (1) is selected from the group consisting of
4-{3-[6-amino-9-((2R,3R,4S,5S)-5-cyclopropylcarbamoyl-3,4-dihydroxytetrahydrofuran-2-yl)-9H-purin-2-yl]-2-propynyl}-piperidine-1-carboxylic acid methyl ester;
4-{3-[6-amino-9-((2R,3R,4S,5S)-5-ethylcarbamoyl-3,4-dihydroxytetrahydrofuran-2-yl)-9H-purin-2-yl]-2-propynyl}-piperidine-1-carboxylic acid methyl ester;
4-{3-[6-amino-9-((2R,3R,4S,5S)-5-ethylcarbamoyl-3,4-dihydroxytetrahydrofuran-2-yl)-9H-purin-2-yl]-2-propynyl}-piperidine-1-carboxylic acid isobutyl ester;
4-{3-[6-amino-9-((2R,3R,4S,5S)-5-ethylcarbamoyl-3,4-dihydroxytetrahydrofuran-2-yl)-9H-purin-2-yl]-2-propynyl}-cyclohexane-1-carboxylic acid methyl ester;
4-{3-[6-amino-9-((2R,3R,4S,5S)-5-ethylcarbamoyl-3,4-dihydroxytetrahydrofuran-2-yl)-9H-purin-2-yl]-2-propynyl}-piperidine-1-carboxylic acid ethyl ester;
4-{3-[6-amino-9-((2R,3R,4S,5S)-5-ethylcarbamoyl-3,4-dihydroxytetrahydrofuran-2-yl)-9H-purin-2-yl]-2-propynyl}-piperidine-1-carboxylic acid isopropyl ester;
4-{3-[6-amino-9-((2R,3R,4S,5S)-5-cyclopropylcarbamoyl-3,4-dihydroxytetrahydrofuran-2-yl)-9H-purin-2-yl]-2-propynyl}-piperidine-1-carboxylic acid 4-fluorophenyl ester;
4-{3-[6-amino-9-((2R,3R,4S,5S)-5-cyclopropylcarbamoyl-3,4-dihydroxytetrahydrofuran-2-yl)-9H-purin-2-yl]-2-propynyl}-piperidine-1-carboxylic acid 2-methoxyphenyl ester;
4-{3-[6-amino-9-((2R,3R,4S,5S)-5-cyclopropylcarbamoyl-3,4-dihydroxytetrahydrofuran-2-yl)-9H-purin-2-yl]-2-propynyl}-piperidine-1-carboxylic acid 4-methoxyphenyl ester;
4-{3-[6-amino-9-((2R,3R,4S,5S)-5-cyclopropylcarbamoyl-3,4-dihydroxytetrahydrofuran-2-yl)-9H-purin-2-yl]-2-propynyl}-piperidine-1-carboxylic acid 3,4-difluorophenyl ester; and
4-{3-[6-amino-9-((2R,3R,4S,5S)-5-cyclopropylcarbamoyl-3,4-dihydroxytetrahydrofuran-2-yl)-9H-purin-2-yl]-2-propynyl}-piperidine-1-carboxylic acid cyclobutyl ester.

10. The method according to claim 6, wherein the compound is administered in a dosage form which is an eyedrop or an ophthalmic ointment.

11-16. (canceled)

17. The composition according to claim 2, wherein the composition is in a dosage form which is an eyedrop or an ophthalmic solution.

18. The composition according to claim 3, wherein the composition is in a dosage form which is an eyedrop or an ophthalmic solution.

19. The composition according to claim 4, wherein the composition is in a dosage form which is an eyedrop or an ophthalmic solution.

20. The method according to claim 7, wherein the compound is administered in a dosage form which is an eyedrop or an ophthalmic ointment.

21. The method according to claim 8, wherein the compound is administered in a dosage form which is an eyedrop or an ophthalmic ointment.

22. The method according to claim 9, wherein the compound is administered in a dosage form which is an eyedrop or an ophthalmic ointment.
